(19) United States
(12) Patent Application Publication
(10) Pub. No.: US 2012/0275950 A1
(43) Pub. Date: Nov. 1, 2012

(54) Scented Vent Clip

(76) Inventors: Amanda Hafner, Oskaloosa, IA (US); Todd Hafner, Oskaloosa, IA (US)

(21) Appl. No.: 13/097,629

(22) Filed: Apr. 29, 2011

(51) Int. Cl. A61L 9/12; A61L 9/04

(52) U.S. Cl. 422/4; 422/123

(57) ABSTRACT

A scented vent clip comprising a spine extending along a top portion of the scented vent clip; a pair of arms connected by the spine, where the pair of arms extend from the spine to a tip at an opposing end; a fragrance layer atop an outer face of the pair of arms, wherein the fragrance layer emits a scent when the scented vent clip is attached to a vent in a room. The method of using the scented vent clip comprises the steps of separating the pair of arms, sliding a vent grate between the pair of arms until the grate touches the spine; and leaving the scented vent clip on the grate to enable air flowing through the vent to pick up the scent and distribute the scent throughout the room.

### BACKGROUND OF THE INVENTION

1. Field of the Invention

The present invention relates to an air freshening device that attaches to the air vent in a room to better circulate the fragrance by utilizing the air pumped through the vent to distribute the pleasing scent.

2. Description of Related Art

Air fresheners are common objects in a home, office, vehicle or store. Essentially anywhere people live, eat, work or play an air freshener or deodorizer may be utilized. The air fresheners may mask odors, refresh stale smells or generally spread a pleasing scent throughout a room. Along with the wide range of locations for use, so are the types of air fresheners available on the market. For example, the air fresheners may be sprays, scented paper cutouts, candles, plug-ins, scented crystals or oils all in scents found pleasing or enjoyable by the user.

While the types of air fresheners are available in a wide variety, they typically concentrate the scent in an isolated area directly around the source of the scent. For example, a plug-in room deodorizer emits a scent but the scent does not travel much beyond the few feet surrounding the outlet. Some plug-ins include small fans to help distribute the scent, but the fans are often too small to make a significant difference in the deodorized area. Conversely, if the scent is more widely distributed the smell is often overwhelming and becomes bothersome instead of helpful. To address this problem, some of the stronger smelling air fresheners have timers built in to decrease the scent impact. But these air fresheners usually require some sort of power source, like batteries or electricity, to control the timer. Therefore, it would be beneficial in the art to provide an air freshener that distributes a scent throughout a room without requiring a power source for circulation.

### SUMMARY OF THE INVENTION

In view of the foregoing disadvantages inherent in the prior art, the general purpose of the present invention is to provide a scented vent clip, configured to include all of the advantages of the prior art, and to overcome the drawbacks inherent therein.

Accordingly, an object of the present invention is to provide a scented vent clip wherein a scented clip is attached to a room vent to evenly distribute a scent throughout a room by utilizing the air passing through the vent for circulation.

To achieve the above object, in an aspect of the present invention, a scented vent clip is disclosed comprising a spine extending along a top portion of the scented vent clip; a pair of arms connected by the spine, where the pair of arms extend from the spine to a tip at an opposing end; a fragrance layer atop an outer face of the pair of arms, wherein the fragrance layer emits a scent when the scented vent clip is attached to a vent in a room. The method of using the scented vent clip comprises the steps of separating the pair of arms, sliding a vent grate between the pair of arms until the grate touches the spine; and leaving the scented vent clip on the grate to enable air flowing through the vent to pick up the scent and distribute the scent throughout the room.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawings, wherein like elements are identified with like symbols, and in which:

FIG. 1 depicts a front view of a scented vent clip in accordance with an exemplary embodiment of the present invention; and

FIG. 2 depicts a side view of a scented vent clip in accordance with an exemplary embodiment of the present invention.

Like reference numerals refer to like parts throughout the description of several views of the drawings.

### DETAILED DESCRIPTION OF THE DRAWINGS

The present invention relates to an air freshening device that attaches to the air vent in a room to better circulate the fragrance by utilizing the air pumped through the vent to distribute the pleasing scent. The present invention describes a scented vent clip to provide a scent delivery apparatus that attaches to any vent on a heating or air-conditioning system where the forced air gently circulates the fragrance throughout the room.

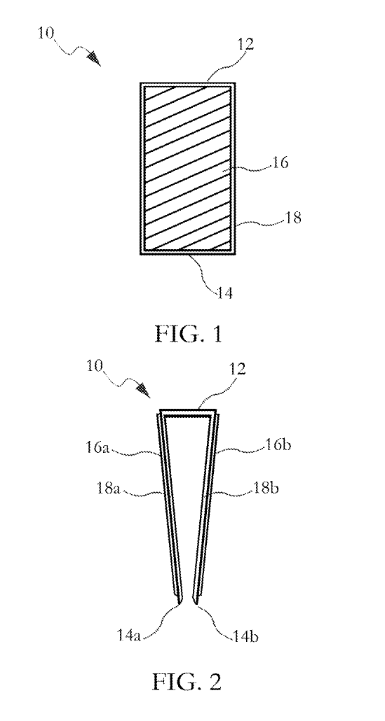

Tuning now descriptively to the drawings, referring to FIG. 1, a front view of a scented vent clip (10) is shown in accordance with an exemplary embodiment of the present invention. The scented vent clip (10) includes a spine (12) at an upper portion of the clip and a tip (14) at a lower portion of the clip. The spine (12) connects a pair of arms (18) (described below) that extend the entire length of the scented vent clip (10).

Along the outer surface of the pair of arms (18) is a fragrance layer (16). The fragrance layer (16) may be a solid gel material, solid powder layer or a scented paper layer. The fragrance layer (16) may be a single, even layer of scent or alternatively it may be a series of panels or pockets distributed along the outer surface of the pair of arms (18). The fragrance layer (16) may be impregnated with a scent like fruit, flowers, leather, laundry, baby powder, spices or another common air freshener variant. Finally, the fragrance layer (16) may be detached from the arm (18) to enable the user to exchange and interchange the scents circulated thereby allowing the user to create their own customized scents. For example the user by attach a coconut fragrance layer on one arm and a pineapple fragrance layer on the other arm for a pina-colada scent, or an apple and cinnamon combination for an apple pie scent. The fragrance layer (16) may be attached to the arm (18) with a hook and loop fastener (commonly known as Velcro), glue, or alternatively it may snap-on into a depression within the arm (18). Referring to FIG. 2, a side view of the scented vent clip (10) is shown in accordance with an exemplary embodiment of the present invention. FIG. 2 better illustrates the pair of arms (18a, 18b) and how they are connected with the spine (12). From the spine (12), the pair of arms (18a, 18b) angle down towards each other until they reach the tips (14a, 14b) at an opposing end. The spine (12) and the pair of arms (18a, 18b) create a triangular shape with a small opening between the tips (14a, 14b). The pair of arms (18a, 18b), when in a relaxed position, angle towards each other and may even touch at the tips (14a, 14b). The pair of arms (18a, 18b), when in an extended position, are separated at the tips (14a, 14b) to receive an object such as a vent grate, panel or the like.

The fragrance layer (16a, 16b) may rest atop the outer surface of the pair of arms (18a, 18b) (as shown). Alternatively, the fragrance layer (16a, 16b) may rest within a depression within the pair of arms (18a, 18b) so that it is flush with the outer surface of the pair of arms (18a, 18b). The fragrance layer (16a, 16b) is a solid material to ensure that when the scented vent clip (10) is transported the fragrance layer (16a, 16b) does not drip, slide or melt from the pair of arms (18a, 18b).

Usage of the scented vent clip is simple and is adaptable into a variety of locations. First the user selects which room to install the scented vent clip and locates the air vent within the room. The user then separates the pair of arms at the tips to enable the scented vent clip to slide onto the vent panel or grate. The pair of arms slide onto the grate until the grate touches the spine. As the furnace or the air conditioning turns on, air blows through the vents thereby picking up scent from the fragrance layer and distributing the scented air throughout the room. This way the fragrance is distributed periodically rather than continuously so that the scent does not become overpowering. The scented vent clip may remain on the grate for up to 30 days until the fragrance layer evaporates or wears away.

The scented vent clip may be made from a durable plastic to ensure that the spine and pair of arms do not bend or distort during use. The dimensions of the scented vent clip range from 1-5 inches in length, ½-3 inches in width and ⅛-¾ inch thick, preferably with the dimensions of 1½ inch in length, 1 inch in width, and ⅓ inch thick. The scented vent clip may be available in a range of colors wherein the color matches the coloring of the vent grate or be colorful for a more decorative appeal, for example when placed in a child's room or nursery.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A scented vent clip comprising:
a spine extending along a top portion of said scented vent clip;
a pair of arms connected by said spine, where said pair of arms extend from said spine to a pair of tips at an opposing end;
a fragrance layer atop an outer surface of said pair of arms, wherein said fragrance layer emits a scent when said scented vent clip is attached to a vent in a room.

2. The scented vent clip according to claim 1, wherein said pair of arms angle towards each other from said spine to said tips to create a triangular shape.

3. The scented vent clip according to claim 1, wherein said scent is one of at least fruit, flowers, leather, laundry, baby powder and spices.

4. The scented vent clip according to claim 1, wherein the dimensions of said scented vent clip are 1½ inch in length, 1 inch wide and ⅓ inch thick.

5. The scented vent clip according to claim 1, wherein said fragrance layer is a solid material.

6. The scented vent clip according to claim 5, wherein said solid material is one of at least a gel, powder or paper.

7. The scented vent clip according to claim 1, wherein said fragrance layer is detachable.

8. The scented vent clip according to claim 7, wherein said fragrance layer is attached to said pair of arms with Velcro.

9. The scented vent clip according to claim 7, wherein said fragrance layer snaps into a depression within said arms.

10. A method of using a scented vent clip comprising the steps of:
separating a pair of arms attached at a spine, where said pair of arms include a fragrance layer;
sliding a vent grate between said pair of arms until said grate touches said spine; and
leaving said scented vent clip on said grate to enable air flowing through said vent to pick up a scent on said fragrance layer and distribute said scent throughout a room.
